(19) United States
(12) Patent Application Publication
(10) Pub. No.: US 2003/0140525 A1
(43) Pub. Date: Jul. 31, 2003

(54) Process for making orthotic insert, an orthotic insert, and a shoe comprising the orthotic insert

(76) Inventor: Robert Michael Branger, Red Lodge, MT (US)

Correspondence Address: HUNTLEY & ASSOCIATES, 1105 NORTH MARKET STREET, P.O. BOX 948, WILMINGTON, DE, 19899-0948, US

(21) Appl. No.: 10/350,885

(22) Filed: Jan. 24, 2003

Related U.S. Application Data
(60) Provisional application No. 60/352,082, filed on Jan. 25, 2002.

(51) Int. Cl. A61F 5/14

(52) U.S. Cl. 36/174; 36/180

(57) ABSTRACT

This will describe unique foot orthotics that are designed to restore, reduce or eliminate foot, ankle, knee, hip and back pain by restoring your natural gait, balance and posture by supporting and cushioning different arches under the foot. These unique orthotics are made in a way that allows different areas of a unitary part to be custom made at different hardnesses by controlling the thickness and cross-linking of the orthotic in that area. This is very beneficial as some parts of the foot need more solid support and other parts need softer support. The parts are made of a chemical-resistant, water-resistant microcellular closed cell material. Also, the orthotics can be made in a variety of overall average stiffness by varying the amount of foaming agent and/or changing materials. This is required for different sporting activities. These orthotics can also be made very light weight by increasing the amount of foaming. These parts can incorporate a cushioning device made with the same process, which further improves the supporting and cushioning of the foot. The orthotic and orthotic/cushioning device combination all slip into the shoe from the top and are replaceable. The present invention also provides for a shoe or sandal further comprising an orthotic of unitary construction, the orthotic being reheatable to conform partially to an individual's foot. The orthotics of the present invention can be made with a high degree of reproducibility and with complicated curves and designs. There are two known processes that can manufacture these parts in the precise reproducible quality needed for orthotics or orthotic/midsole combinations.

### CROSS-REFERENCE TO RELATED APPLICATION

This application claims the benefit of U.S. Provisional Application Serial No. 60/352,082, filed Jan. 25, 2002.

### BACKGROUND OF THE INVENTION

An orthotic is defined as a device or devices designed to help to reduce or eliminate pain or discomfort by helping to restore a more natural gait, balance and/or posture by supporting and cushioning different parts of the foot, and thus the body. Specifically, foot orthotics are designed to reduce or eliminate foot, ankle, knee, hip and back pain by restoring your natural gait, balance and posture by supporting and/or cushioning different points under the foot or the foot as a whole. Various orthotic devices have been commercially available for years. Similarly, there are a number of different manufacturing methods and materials that have been used to make foot orthotics. Orthotics have been made of solid and foamed polymers. Foamed orthotics are typically made with open cell polyurethane materials, which show uniform cell structure and consistent hardness throughout the part. These known foamed orthotics are typically made by machining or skiving the orthotic from a larger piece of foam. They can also be made with a poured polyurethane process where polyurethane is poured into a mold and the foaming takes place to fill the mold. Open cell materials, including polyurethane, are problematic as orthotic materials because these materials, especially polyurethane, will break down when exposed to sweat or water.

Also, orthotics that have varying hardnesses throughout can be manufactured by cementing foamed pieces of differing hardnesses together. The individual parts of such orthotics are each foamed to different densities or made from different materials that give them the different hardnesses. When the parts are combined to make the orthotic, there is not a smooth hardness transition between the different areas. Also, the glue used to attach the different parts creates unwanted stiffness and rigidity due to the hardness of the bonding agent, as well as being susceptible to breakdown.

Other orthotics can be made by casting a person's foot, and using the cast as a template for the orthotic which can then be made from solid plastic which is thermoformed or injected molded to mimic the foot. Injection molded parts made with this process use the conventional injection molding process where a solid part results, cold molds are used, and no chemical reaction takes place during the injection molding process. Even other orthotics are made from cork held together by a polymer or other type of binding agent. Orthotics can also be made by combining different materials like gel heel pads and foam parts, such as a gel heel pad surrounded by a compression molded foamed polyolefin or foamed polyurethane. Orthotics have also been made with polymer films welded together to enclose pockets of air supporting and cushioning different areas of the foot. The main purpose of all these orthotics is to reduce or eliminate foot, ankle, knee, hip and back pain by restoring your natural gait, balance and posture by supporting and cushioning different points or arches under the foot. A need remains, however, for an orthotic device of unitary construction that has areas of differing hardness and that resists the negative effects associated with prior materials when exposed to water or sweat.

Most orthotics slip into the top of the shoe. There exists a foamed polyurethane cushioning device which slips into the top of the shoe but this part doesn't incorporate any orthotic support and is made of open cell material which will break down under long term exposure to water or sweat. This part could be considered a midsole (cushioning part of a sport shoe) since it had thickness in the heel and forefoot that is similar to the thickness offered in sports shoes that have cushioning midsoles. Most sport shoes incorporate an insole, or sockliner, which is a uniform or slightly tapered thickness, usually 3 to 6 mm and is inserted into the top of the shoe. The typical sockliner has a felt, textile, leather or other liner laminated to the top of it to allow the foot to slip into the shoe easily. Most sockliners are made from die-cut compression molded foamed ethylene vinyl acetate copolymer or polyurethane foam. Some can be made from Neoprene foam, leather or other materials. These sockliners typically offer little or no arch support. Some are molded with a simple bend in the arch area so the foam extends up alongside the inside of the shoe. Reproducibility of die-cut or compression molded foamed ethylene vinyl acetate copolymer sockliners is difficult and parts vary widely due to the nature of the manufacturing process. Shoes have been made with a cork polymer blend that inserts into the top of the shoe. These inserts incorporate arch support but offer little or no cushioning, are unnecessarily heavy, and can't be made in different hardnesses.

Multiple layer sockliners have also been made where the sockliner was heated to a softening point and then molded by placing the foot on it and applying pressure. In some of these cases, the sockliner could be classified as an orthotic since it offered support due to a stiff layer incorporated into the part. The molding is typically done in the shoe after the sockliner has been heated with a heat gun, or other suitable means. All these heat formable orthotics are made with a system of layers where each layer functions differently. When layered, at least 2 layers are always used where at least one layer is a moldable layer. In other cases more layers are added to offer insulation or perform other functions. These multi-layer parts are very complicated to make and it is difficult to manufacture them with uniform, repeatable properties as described herein.

Another method for forming sockliners is making them so they incorporate susceptor-impregnated ingredients which allows them to be microwaveable. When microwaveable, they need to incorporate excess ingredients and process steps adding to the complexity of the process and additional cost. Also, some of the susceptor-impregnated ingredients can be hazardous. There is a need for a simple unitary, foamed orthotic which can offer support to the foot, offer cushioning, offer insulation, be made in a reproducible manufacturing process, and not require susceptor-impregnated ingredients.

Accordingly, sockliners and inserts fail to provide the advantages of the present invention, and are subject to the following shortcomings: Prior orthotic devices break down when exposed to moisture or sweat, and thus lose their ability to cushion; prior orthotics that provided different hardnesses in different parts of the orthotic were not of unitary construction, and thus were affected by the adhesive that connected the different parts, and the transition between parts of different hardnesses was not smooth; use of adhesives causes an abrupt hardness shift at the glue line, unwanted rigidity, and can decrease the life of the orthotic because of the tendency of adhesives to break down, allowing the parts of the orthotic to come apart.

### SUMMARY OF INVENTION

The present invention provides a process for making a foot orthotic for a shoe, boot, sandal, or sneaker or sport shoe, an orthotic insert made according to the process, and a shoe containing the orthotic insert. The present invention provides orthotic devices having different levels of support or cushioning, while being constructed of a single piece. The present invention provides a process for manufacturing such orthotic devices that allows for varying the materials, amount of foaming, and/or the amount of cross-linking during the manufacturing process, and thus allows for the control of the hardness of the resulting orthotic.

The present invention provides a means for producing orthotic devices that can incorporate the midsole and at the same time offer orthotic support. The present invention provides a means of producing high quality foamed orthotic devices that are reproducible, as well as providing high quality orthotic devices which can be foamed to a low density.

Specifically, the present invention provides a process for fabricating an orthotic insert for use in footwear, the process comprising: Compounding a foamable cross-linkable compound giving the required physical properties; Mixing the compound at temperatures below the cross-linking and blowing temperature; Injecting the compound into or onto a hot mold using precise process controls, which allow the exact amount of molten (plasticized) polymer to be utilized and injected at a precise speed, temperature, weight and pressure to allow a quality finished part to be produced; Heating the foamable composition to above the decomposition temperature of the blowing agent and above the activation temperature of the cross-linking agent; Controlling the temperature; Holding the mold closed until the cross-linking and foaming reactions take place, and; Opening the mold allowing the finished part to explode or quickly expand out of the mold.

The present invention also provides a process for forming an orthotic shoe insert comprising: Compounding a foamable cross-linkable compound to obtain the required physical properties; Heating and mixing the compound at temperatures below the cross-linking and blowing temperature; Injecting the compound into or onto a hot mold using precise process controls; Regulating the exact amount of molten (plasticized) polymer to be utilized; Regulating the speed at which the compound is injected; Regulating the temperature at which the compound is injected; Regulating the pressure at which the compound is injected; Heating the compound within the mold to a temperature higher than the decomposition temperature of the blowing agent and above the activation temperature of the cross-linking agent; Controlling the temperature; Holding the mold closed until the cross-linking and foaming reactions take place, and; Opening the mold allowing the finished part to explode or quickly expand out of the mold.

The present invention further provides a shoe or sandal comprising a sole and an upper connected to the sole, wherein the shoe is adapted for insertion of an orthotic according to the present invention, as well as a shoe wherein the orthotic further comprises a midsoles where the midsoles and orthotic are a unitary piece.

The present invention also provides orthotic inserts made according to the processes disclosed herein.

The present invention also provides for a shoe or sandal, wherein the shoe is adapted for insertion of an orthotic according to the present invention, and orthotic inserts are made according to the processes disclosed herein, which can be reheated in a conventional oven, taken out after a specified period of time and stepped upon to conform partially to an individuals foot.

### BRIEF DESCRIPTION OF THE DRAWINGS

FIG. 1 shows two cross-sections of the orthotic described in the present invention. FIG. 2 shows a typical centerline cross-section of the orthotic described in the present invention.

FIG. 3 shows a top view and a side view of an orthotic of the present invention.

FIG. 4 represents the side view of a last FIG. 5 is a side view of a last with the shaded portion being the material added to the last to extend it down further. This shaded portion can also represent the unitary midsole/orthotic side view.

FIG. 6 is a back view of a last.

FIG. 7 is a back view of a last with the shaded portion being the material added to the last to extend it down further. This shaded portion can also represent the unitary midsole/orthotic back view.

FIG. 8 is a cross section of an orthotic/midsole combination of the present invention

### DETAILED DESCRIPTION OF THE INVENTION

The present process provides a means of fabricating an orthotic insert for use in a shoe, an orthotic insert, a shoe or sandal containing the orthotic insert. The present invention also provides a combination orthotic/midsole of unitary construction. The combination orthotic midsole, as well as the orthotic of the present invention can be reheated and partially molded to conform to the contours of a person's foot. Regardless of the application of the orthotic, it can have the following characteristics. The orthotics of the present invention can be accurately reproduced by the process of the present invention, and can be used as replaceable parts having exact dimensions. For example, orthotics produced according to the present invention can have a variation of dimensions of +/−0.75 mm over the total length, +/−0.50 mm in width, and +/−0.3mm thickness of a full length orthotic for a men's size 9 shoe. The density of the orthotics, and the density variation within each orthotic of the present invention is also accurately reproducible. Specifically, the present invention provides for orthotics and parts therefore having an overall density as low as 0.10 gm/cc and as high as 0.90 gm/cc with typical overall part densities in the range of 0.15 gm/cc to 0.85 gm/cc.

Similarly, depending on the application for which the orthotic of the present invention is used, the orthotics can have hardness differences in different areas of a single foot orthotic device based on the cross-sectional design. Hardness variations will be similar to those demonstrated in Table 1 and/or Table 2. Any of these hardness values can be controlled to vary by as little as +/−5%. In the present invention, hardness is inversely proportional to the thickness of the foam. The thicker the foam, the softer that portion of the orthotic. This variation in hardnesses that is a key aspect of the present invention provides a means of giving support to the different points, or arches, under the foot.

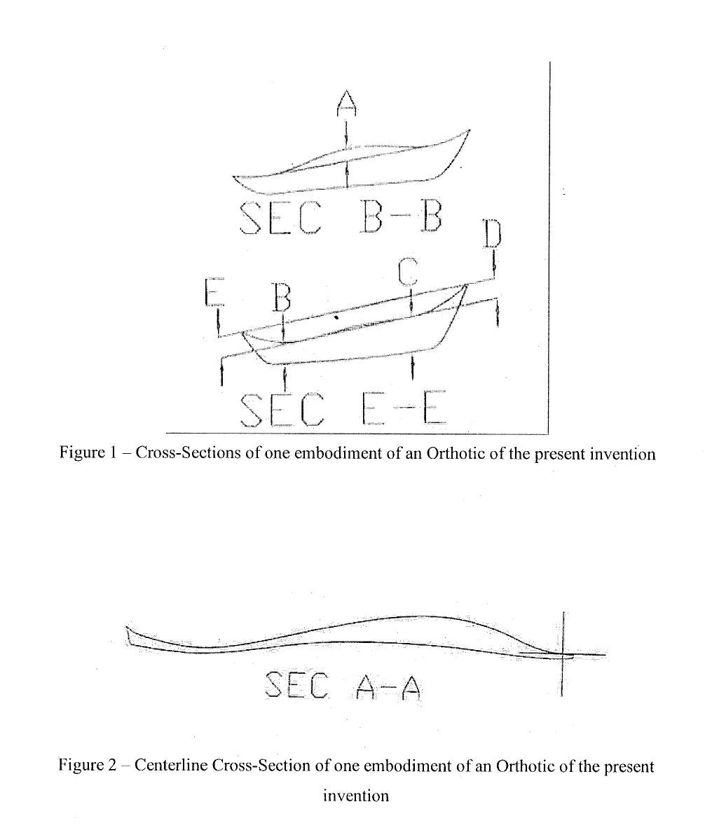

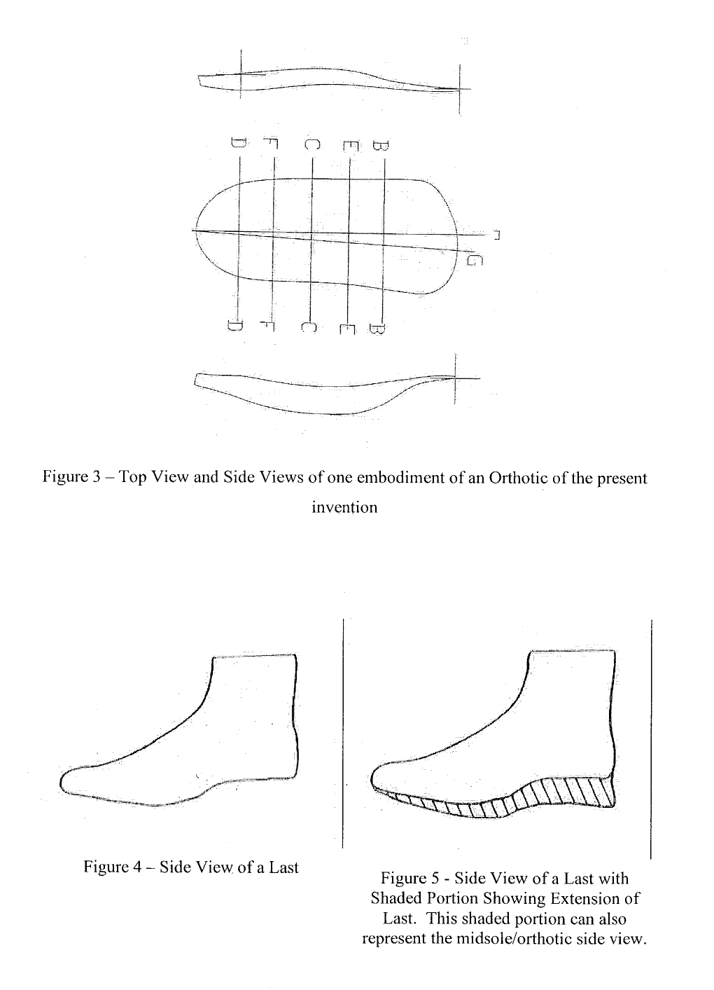

The present invention provides an orthotic, and a means of making such an orthotic, wherein the orthotic is designed to reduce or eliminate foot, ankle, knee, hip and back pain by restoring your natural gait, balance and posture by supporting and cushioning different arches under the foot. In the present invention, an orthotic of the present invention is shown in FIGS. 1-3. Preferred embodiments of the orthotic of the present invention can be defined where there is at least one of the following: Metatarsal support as shown by dimension A in Section B-B; Medial arch support as shown by the difference between dimension C and dimension B in Section E-E; And media and lateral support are shown by dimension D and E in Section E-E. In the figures, the right side is the medial side and the left side is the lateral side of the foot. Preferred embodiments of the orthotics of the present invention can also be defined where there is at least one of the following: Metatarsal support where Dimension A is a minimum of 1 mm for a men's size 9; Medial arch support where Dimension C is a minimum of 1 mm more than Dimension B for a men's size 9; Medial arch support where Dimension D is a minimum of 1 mm for a men's size 9; Lateral arch support where Dimension F is a minimum of 1 mm for a men's size 9.

The present invention provides a means to produce an orthotic, which could incorporate the midsole and orthotic as one unitary piece, which slips in the top of the shoe. Very complicated 3-dimensional parts are required for this application. These parts can be described as the shaded portion of FIG. 5 and FIG. 7. Basically, the midsole/orthotic combination has to fit precisely into the cavity formed by the extended last (shaded portion of FIG. 5 and FIG. 7) in the shoe manufacturing process. Therefore, the shaded portions in FIG. 5 and FIG. 7 can represent either 1) The extended last used in the shoe manufacturing process to leave a cavity to house the midsole/orthotic combination or 2) The actual midsole/orthotic combination that slips into this cavity. Also, to further describe this invention, the midsole/orthotic combination must incorporate the description of an orthotic as shown in FIGS. 1-3. The midsole/orthotic combination is made as one integral piece. The orthotic portion of the midsoles/orthotic combination is described according to the specifications herein.

The present invention provides a means of producing an orthotic that can be custom made for requirements of sports, which require different overall average stiffness of orthotic support. For example a walking shoe might require more softness in an orthotic or midsole/orthotic combination than a basketball shoe due to impact forces. A walking shoe orthotic or midsole/orthotic combination may be made of Material A in Table 1 while a basketball shoe orthotic or midsole/orthotic combination may utilize Material C in Table 2.

The present invention provides a process that allows for the production of an orthotic of different overall average stiffness which can utilize the softer orthotic for a break-in period until the foot gets used to it. After the foot becomes accustomed to the softer orthotic, a harder orthotic can be used. The present invention provides a means of producing orthotics having an average overall hardness range from 25 Asker C to 90 Shore A with a typical range of 45 Asker C to 70 Shore A. (These measurements would be made near the part surface with the skin machined off to allow accurate measurement.)

The present invention also provides for a shoe or sandal, wherein the shoe is adapted for insertion of an orthotic according to the present invention, and orthotic inserts are made according to the processes disclosed herein, which can be reheated in an oven at a later date, taken out after a specified period of time and stepped upon to conform partially to an individuals foot.

This invention provides replaceable foamed foot orthotics that can be made in a way that provides cushioning and support in a one-piece unit that is inserted into the top of the shoe. Unique properties of the orthotics include the ability to design different hardnesses within the unit by varying the thickness of the part. This is critical, as some parts of the foot require more support while other parts require more cushioning. Typically, the medial arch of the foot requires more support while the center of the heel requires more cushioning. This can be accomplished by making a midsole/orthotic combination where the heel is thicker than the arch area. It is also desirable to have more cushioning in the metatarsal area which can be incorporated by making the metatarsal area thicker than the area supporting the arch. This is demonstrated by Section B-B in FIG. 1. Data provided in Table 1 and Table 2 show examples of different hardnesses for different cross-sectional thickness of the orthotic. Hardness ranges from 25 Asker C to 90 Shore A with a typical range of 45 Asker C to 70 Shore A are used. As can be seen, a wide range of hardnesses can be utilized in a single orthotic by varying the thickness. Shortening or lengthening the cross-linking time or temperature can control hardness variation. This effectively controls the amount of cure or cross-linking. Longer cross-linking times and/or higher temperatures reduce the cross-sectional hardness variation and conversely, shorter cross-linking times and/or lower cure temperatures increase the cross-sectional hardness variation. Table 1 gives an example of hardness variation with more cross-linking and Table 2 gives an example of hardness variation with less cross-linking. These hardness values can be further changed by varying the amount of cross-linking. Hardness measurements should be taken at least 3 different times and averaged in order to minimize measurement error.

TABLE 1
Hardness versus Thickness
More Cross-linking
Hardness measurements are taken at the center of the listed thicknesses

| Material A | | Material B | | Material C | | Material D | |
|---|---|---|---|---|---|---|---|
| Cross- | | Cross- | | Cross- | | Cross- | |
| sectional | | sectional | | sectional | | sectional | |
| Thickness | Hardness | Thickness | Hardness | Thickness | Hardness | Thickness | Hardness |
| (mm) | (Asker C) | (mm) | (Asker C) | (mm) | (Asker C) | (mm) | (Shore A) |
| 25 | 30 | 25 | 44 | 25 | 54 | 25 | 63 |
| 12 | 33 | 12 | 48 | 12 | 59 | 12 | 69 |
| 6 | 38 | 6 | 52 | 6 | 65 | 6 | 74 |
| 3 | 42 | 3 | 55 | 3 | 70 | 3 | 78 |
| 2 | 46 | 2 | 59 | 2 | 78 | 2 | 84 |

TABLE 2
Hardness versus Thickness
Less Cross-linking
Hardness measurements are taken at the center of the listed thicknesses

| Material A | | Material B | | Material C | | Material D | |
|---|---|---|---|---|---|---|---|
| Cross- | | Cross- | | Cross- | | Cross- | |
| sectional | | sectional | | sectional | | sectional | |
| Thickness | Hardness | Thickness | Hardness | Thickness | Hardness | Thickness | Hardness |
| (mm) | (Asker C) | (mm) | (Asker C) | (mm) | (Asker C) | (mm) | (Shore A) |
| 25 | 25 | 25 | 39 | 25 | 49 | 25 | 58 |
| 12 | 29 | 12 | 44 | 12 | 55 | 12 | 65 |
| 6 | 35 | 6 | 49 | 6 | 62 | 6 | 71 |
| 3 | 40 | 3 | 53 | 3 | 68 | 3 | 76 |
| 2 | 46 | 2 | 59 | 2 | 78 | 2 | 84 |

In the above table all materials are described by a foamable composition which is prepared by formulating a ethylene vinyl acetate copolymer compound. The copolymer is blended in a kneader mixer with 1.5 parts azodicarbonamid blowing agent, and 0.9 parts dicumal peroxide cross-linking agent, all based on 100 parts of the ethylene vinyl acetate resin. Other ingredients like stearic acid can be added to aid in processing without significantly changing the physical outcome of the materials. Material A is represented by an ethylene vinyl acetate copolymer with 30% vinyl acetate and 70% ethylene, Material B is represented by an ethylene vinyl acetate copolymer with 22% vinyl acetate and 78% ethylene, Material C is represented by an ethylene vinyl acetate copolymer with 15% vinyl acetate and 85% ethylene, and Material D is represented by an ethylene vinyl acetate copolymer with 12% vinyl acetate and 88% ethylene.

The orthotics can be made in a repeatable process, which provides parts that can be of high quality and reproducibility. This is critical since a foot orthotic must fit the base of the foot in a consistent way or foot pain won't be eliminated and may actually be aggravated. In the present invention, reproducible dimensions can be held to +/−0.75 mm over the total length, +/−0.5 mm in width, and +/−0.3mm thickness of a full length orthotic for a men's size 9 shoe. In the thicker cross-sections of the part the part is softer when compared to the thinner cross-sections. The precise process controls allow parts to be reproduced consistently. Hardness reproducibility in any area of the part can be controlled to +/−5%.

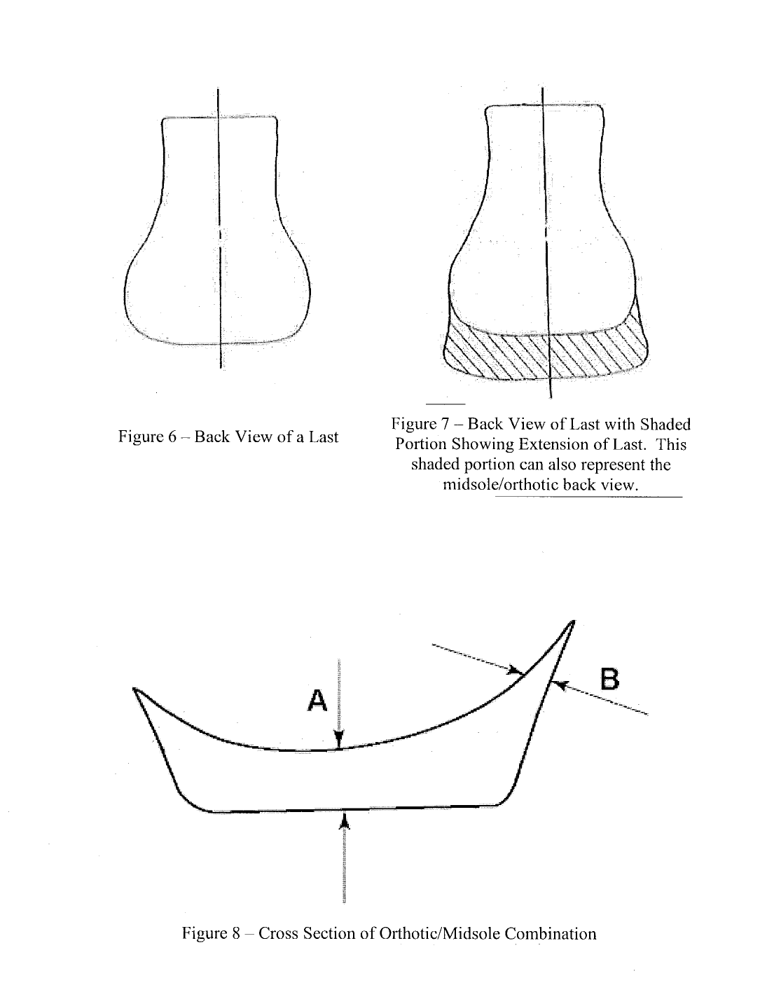

Also, this invention can combine an orthotic and the cushioning part of a typical sport or work shoe, usually called a midsole, in one piece, which will slip into the top of the shoe if the shoe construction has been designed to accommodate the orthotic/midsole combination. These parts are replaceable which is important since midsoles have a limited life and usually a sport shoe like a running shoe is discarded after the cushioning characteristics of the midsole deteriorate, usually after 300-500 miles in a running shoe. A textile material like felt may be laminated on top of the unit to allow the sock or foot to slip easily into the shoe. The sock or bare foot is designed to come in direct contact with the orthotic or orthotic/midsole or in contact with the laminate on top of the unit. A sockliner could be optionally placed on top of the orthotic. In the case where the orthotic/midsole combination is used, the shoe would have to be constructed in a way that allows a deeper cavity inside the shoe so when the orthotic/midsole slips into the top of the shoe, the cavity is filled up properly to allow functionality of the shoe. One way to make a shoe like this would be to utilize a shoe last that has been extended on the bottom of the last and then when the shoe upper is formed around the last, the cavity inside the shoe is larger than that of a typical shoe. For the midsole/orthotic to fit into this cavity properly, precise size and dimensional control must be exercised during the manufacture of the insert or the insert won't fit into the complicated 3 dimensional cavity inside the shoe. FIGS. 6 and 7 show sketches of a back view of a last. FIG. 6 shows the typical last and FIG. 7 shows how the last would need to have material added in order to make the shoe cavity deeper to accommodate the orthotic/midsole combination. FIGS. 4 and 5 show similar sketches but they represent the side view of the last. In this example, the orthotic/midsole combination would have to be the same shape as the shaded parts of FIG. 5 and 7 in order to fit into the shoe properly. A typical orthotic/midsole combination would be, but not limited to, 10 to 25 mm thick in the center of the heel to 5 to 15 mm thick in the middle of the forefoot under the ball of the foot with arch support built into the medial side, and having arch support directly behind the ball of the foot along the centerline of the foot, also called the metatarsal area. Other support could include arch support in the lateral side and a heel cup to support and stabilize the heel. There are many other ways the shoe could be constructed. For example, a second piece made of plastic, wood, or other material could be placed under a custom last to extend it, or the actual midsole/orthotic combination could be placed under the custom last. The upper of the shoe would be constructed around any of these combinations. In some cases, a rim of foamed EVA (ethylene vinyl acetate copolymer) or other material may be placed around the perimeter of the top of the outsole to support the overall shoe construction. This rim would simply replace the outside portion of the orthotic/midsole combination. Again, a cavity would still be formed for the insertion of the orthotic/midsole combination, it would simply be slightly smaller due to the space taken up by the rim. Still another way to construct the shoe would be to stitch the bottom of the shoe to the upper of the shoe where the bottom of the shoe is basically the midsole/orthotic combination. This method is currently used in the constructions of many types of boots with conventional bottom units.

The present orthotics are all made of a foamable polymer, which produces a chemical, water, and sweat resistant part with microcellular closed cells. This is important as it doesn't retain water and sweat like an open cell foam would and will not break down from water or sweat like many other polymers such as polyurethane. These orthotics can be made in light weights, down to an overall density of 0.10 gm/cc for the lightest range and some of the heavier parts may have an overall density up to 0.90 gm/cc which is still much lighter than most solid orthotics. Typical average part densities range from 0.15 gm/cc to 0.85 gm/cc depending on the hardness required of the finished part. In some cases, stiffer, harder parts are required which would utilize higher densities. This may be for but not limited to sports where minimum flexibility and energy loss is desired in the shoe/orthotic combination like in-line skating or bicycling. An activity like walking may require a softer shoe/orthotic combination, which could utilize lower density parts. Also, a softer orthotic may be used for a break-in period until the foot becomes used to the new orthotics. Another way of changing overall average stiffness would be to utilize different polymers in the initial compounding process. Typically harder polymers in their natural state will produce harder orthotics in the foamed state when compared to softer polymers foamed to the same density.

Also, many orthotics take time to get used to. Another benefit of this invention would be to manufacture a part that is softer for initial break-in and then progress to stiffer, harder orthotics of the same dimensions as the foot gets used to the new support device.

This invention can be manufactured by: (1) Compounding a foamable cross-linkable compound giving the required physical properties where the compound is mixed at temperatures below the cross-linking and blowing temperature; (2) Injecting the compound into or onto a hot mold using precise process controls, which allow the exact amount of molten (plasticizied) polymer to be utilized and injected at a precise speed, temperature, weight and pressure to allow a quality finished part to be produced; (2A) In one case the molds are closed during injection and a vacuum assists the flow of polymer compound into the hot mold through vacuum ports at the back of the mold cavity. This process is more typical for compounds using a majority of thermoplastic polymers like EVA (ethylene vinyl acetate copolymer) or polyethylene, which have a low viscosity allowing the flow of polymer to fill the closed mold; (2B) In a second case, the molds are open and the plasticized, compound drools onto a hot mold using a movable injector which assures the correct amount of compound is deposited in the correct location to fill the mold. After filling the mold, the injector moves away. The mold is then closed. This process is more typical for thermosetting elastomer compounds that have high viscosity preventing easy flow into a closed mold; (3) Heating the foamable composition to above the decomposition temperature of the blowing agent and above the activation temperature of the cross-linking agent. Very precise temperature controllers are used on the molds to make sure parts are reproducible; (4) Holding the mold closed until the cross-linking and foaming reactions take place; (5) Quickly opening the mold allowing the finished part to explode or quickly expand out of the mold

The present invention is applicable to the preparation of foamed foot orthotics made of a wide variety of polymers, including, for example, polyolefins such as polyethylene and polypropylene; ethylene copolymers and terpolymers such as ethylene vinyl acetate copolymer, ethylene methalcrylic acid copolymers; thermoplastic and thermosetting elastomers; polyesters such as polyethylene terephthylate and polybutylene terephthylate; as well as blends and alloys of two or more of such polymers. Polyolefins, ethylene vinyl acetate copolymers, and thermosetting elastomers and their blends have been found to be particularly suitable for the instant process.

A foamable composition can be prepared by blending the polymer to be foamed with cross-linking agent and blowing agent. Cross-linking agents that can be used will depend on the particular polymer used in the foaming process and the desired hardness of the finished product but can include peroxide including Dibenzoyl peroxide, t-butyl peroxybenzoate, 1,1-Di-(t-butylperoxy)-3,3,5-trimethylcyclohexane, Dicumyl peroxide, Di-(2-t-butylperoxyisopropyl)benzene, t-butylcumylperoxide, Di-t-butylperoxide and sulfur or sulfur donor cross-linking agents. Of these, dicumal peroxide has been found to be particularly satisfactory with the preferred polyolefins, ethylene vinyl acetate copolymers, and thermosetting elastomers. Typically, the cross-linking agent will be used in quantities of about from 0.3 to 3.0 parts per 100 parts of the polymer resin.

Blowing agents that can be used in the present invention will similarly depend on the particular polymer being foamed and the desired hardness of the finished part. Representative blowing agents which can be used include azobisisobutyronitrile, azodicarbonamid, p-toluene sulfonylhydrazide, 4,4′-oxybis (benzenesulfonyhydrazide), N,N-dinitrosopentamethylenetetramine and sodium bicarbonate. Still other blowing agents that can be used in the instant invention include modified azodicarbonamids, hydrazides, and 5-phenyltetrazole.

The amount of blowing agent used will in part depend on the desired density and hardness of the final foamed product. However in general, the amount of blowing agent will be about from 0.5 to 10 parts of blowing agent per 100 parts of the polymer resin. The foamable composition is typically also formulated with blowing coagents to reduce the cycle time by increasing cross-linking rate, blowing agent activator release agents and fillers, as will be recognized by those skilled in the art of preparing foamed articles. Typical release agents include zinc stearate and stearic acid. Zinc oxide is often used as a blowing agent activator but is not necessary for the invention to work.

The components of the foamable composition can be blended by any suitable means, for example, a high shear mixer or kneader such as a Banbury mixer. Extrusion apparatus can also be used for blending, such as conventional twin-screw extruders. The blended material can be either pelletizied in a further downstream operation or cut into strips that can then be fed into the injector of the molding machine. All these blending methods take place below the activation temperature of the blowing agent and cross-linking agent.

In accordance with the instant process, the foamable composition is introduced into a mold that is heated to a temperature higher than the decomposition temperature of the particular blowing agent used and higher than the activation temperature of the cross-linking agent. While this temperature will vary with the particular blowing agent selected, it should be at least about 145 degrees C., for example, for the preferred modified azodicarbonamid blowing agents. Typically, the blowing agent and the cross-linking agent are selected to have substantially the same decomposition and activation temperature, respectively.

In the instant process, the compound polymer, either in strip or pellet form, is fed into the feed section of the injection machine with a proper screw, usually a low shear screw, and very precise temperature controls on the different heating zones of the barrel of the machine. In some cases, a mixing section is added to the screw to allow color or other additives to be added to the compound. The material is plasticizied in the machine and either held in front of the screw or in an accumulator or piston while waiting to be injected or drooled in the mold. Precise temperature control is used on the nozzle and/or piston or accumulator if used. This temperature is below the activation temperature of the blowing agent and cross-linking agent.

In the closed mold method, the nozzle of the injector is placed against the hot mold creating a seal and a vacuum may be pulled on the mold to assist mold filling and eliminate any oxygen, which might be present, which could cause burning of the finished product. Precise machine controls inject the exact amount of plastizied compound into the mold at a preset speed to fill the mold 100% or nearly 100%. In most cases, a moveable gate will close off the runner of the mold preventing back-flow of the polymer and the nozzle moves back away from the mold. The nozzle moves away to prepare to move to a different mold on another station and/or to prevent the compound from cross-linking or blowing in the nozzle since the nozzle is at a much lower temperature than the mold. A timer is started when the mold is filled or at some other consistent point like when the injection starts.

In the open mold method, the nozzle of the injector is placed above and towards the end of the open mold cavity. Using precise controls, the material is drooled into the open mold while the injector moves back along the mold cavity drooling the material in the full length of the mold filling the mold to slightly more than 100%. The injector then moves out of the way and the mold is closed. Any excess material will be squeezed out of the mold as flash. A timer is started once the mold is closed or at some other consistent point like when the injection starts.

A predetermined cure time has been set in both cases and when the cure time has expired, the mold quickly opens and the finished part explodes or quickly expands out of the mold. Sometimes an external mold release is applied to the mold before the polymer is injected to assist in the release of the parts. Also, internal mold releases known to those skilled in the arts may be compounded into the material. The reason the part explodes or expands out of the mold is due to the blowing agent that has been activated during the curing process. Pressure of a closed mold has kept the gases produced by decomposition of the blowing agent in solution but when the pressure is released due to a quickly opening mold, the gas expands making the part expand out of the mold.

The present invention is further illustrated by the following specific examples. A foamable composition is prepared by formulating ethylene vinyl acetate copolymer containing 15% vinyl acetate and 85% ethylene. This copolymer is blended in a kneader mixer with 1.5 parts azodicarbonamid blowing agent, 0.9 parts dicumal peroxide cross-linking agent and 0.5 parts stearic acid for a processing aid, all based on 100 parts of the ethylene vinyl acetate resin. The resulting compound is removed from the kneader mixer after the polymer temperature reaches 95 to 130° C. and placed on a 2-roll mill. The polymer is kept warm on the roll mill and pieces of the plastizied polymer are cut off and fed into an extruder, which pelletizes the compound, which is then cooled and dried.

The process uses an injection machine, usually with a compression ratio screw of less than 1.7 to 1. Barrel and nozzle temperatures are set at around 90 degrees C. The compounded polymeric material will have a melt temperature of 100 to 130 degrees in the present example. Other polymers could exhibit higher or lower melt temperatures. Polymers with higher melt or softening temperatures will have higher melt temperatures exiting the machine. This melt temperature is controlled by screw RPM, screw design, injection backpressure during loading of the screw, heating zones and injection pressure. The material is injected into a hot mold that has a temperature of 175° C. During injection, a vacuum is pulled on the mold to assist filling and eliminate any oxygen which could cause burning of the part due to the high mold temperature, shear heat generated through friction caused by the filling action of the mold sprue, gate, runners, cavity, and the melt temperature of the compound before it enters the mold. After injection a moveable gate closes off the runner to prevent back flow of the polymer and the injector moves away from the mold to prepare for another injection in another mold at a different injection station or it simply moves away to keep the nozzle away from the hot mold to keep the material from cross-linking in the nozzle.

The process of the present invention should be controlled so that the average temperature in the mold varies by less than 1 degree centigrade. If the temperature varies more than this, the part won't have consistent size or hardness properties and is not usable.

After the material remains in the closed mold for 400 seconds, the mold quickly opens and the part explodes out of the mold. The part is then placed on a cooling rack inside a controlled temperature cooling tunnel and cooled at a uniform temperature of 60 degrees centigrade for 40 minutes.

The final part will be about 1.25 times larger then the mold cavity in the three dimensions, length, width, and height. Reproducibility will be as follows:

+/−0.5 gram overall weight.

+/−0.75 mm total length for a size 9 men's shoe size.

Hardness profile as that shown in Material C in Table 1 with a variation of+/−5% of the given value.

In the second example, everything is held the same as the first example but the material is held in the closed mold for 350 seconds instead of 400 seconds.

The final part will be about 1.25 times larger then the mold cavity in the three dimensions, length, width, and height. Reproducibility will be as follows:

+/−0.5 gram overall weight.

+/−0.75 mm total length for a size 9 men's shoe size.

Hardness profile as that shown in Material C in Table 2 with a variation of +/−5% of the given value.

In the third example, everything is held the same as the first example but the hot mold temperature is set at 165 degrees C. instead of 1-75 degrees C.

The final part will be about 1.25 times larger then the mold cavity in the three dimensions, length, width, and height. Reproducibility will be as follows:

+/−0.5 gram overall weight.

+/−0.75 mm total length for a size 9 men's shoe size.

Hardness profile as that shown in Material C in Table 2 with a variation of+/−5% of the given value.

Using the process in the above examples, orthotics could be made where the orthotic or orthotic/midsole combination can be placed in an oven at 93 degrees centigrade, heated for 4 minutes, taken out of the oven, placed in the shoe, and stood on for 1 minute. This will partially conform the insert to the shape of the foot. In another example, the orthotic or orthotic/midsole combination can be placed in an oven at 120 degrees centigrade, heated for 1 ½ minutes, taken out of the oven, placed in the shoe, and stood on for 1 minute. This will partially conform the insert to the shape of the foot.

In one example of an insertable midsole/orthotic combination, the parts are made in the shape of the shaded portion of FIGS. 5 and 7. In this example, a men's size 11, the heel thickness is 22 mm and the forefoot thickness is 15 mm. The cross-section of these parts in the arch area of the foot are represented in FIG. 1 where Dimension A is 4 mm; The difference between Dimension B and C is 4 mm; And also in FIG. 1, Dimension E is 7 mm and Dimension D is 7 mm.

In another example of an insertable orthotic, the part will have the shape of FIGS. 1, 2 and 3. In this example, a men's size 11, the heel thickness is 2 mm and the forefoot thickness is 0 mm as this insert is only 60% as long as the foot and doesn't extend to the forefoot. In FIG. 1, Dimension A is 4 mm; The difference between Dimension B and C is 4 mm; And Dimension E is 7 mm and Dimension D is 7 mm.

The following 2 examples show how hardness is inversely proportional to thickness and how this hardness can be controlled. For both of these examples, an orthotic/midsole is shown in FIG. 8 where Dimension A is 20 mm and Dimension B is 5 mm.

### EXAMPLE 1

Referring to Table 2 (Hardness versus Thickness—Less Cross-linking), Material A would have the following properties:

The hardness of a point in the center of thickness A is approximately 27 Asker C.

The hardness of a point in the center of thickness B is approximately 38 Asker C.

Due to the differences in hardness, this orthotic/midsole will offer more support across Dimension B and more cushioning across Dimension A.

### EXAMPLE 2

Referring to Table 2 (Hardness versus Thickness—More Cross-Linking) Material A would have the following properties:

The hardness of a point in the center of thickness A is approximately 31 Asker C.

The hardness of a point in the center of thickness B is approximately 39 Asker C.

Shortening or lengthening the cross-linking time or temperature can control hardness variation. This effectively controls the amount of cure or cross-linking. Longer cross-linking times and/or higher temperatures reduce the cross-sectional hardness variation and conversely, shorter cross-linking times and/or lower cure temperatures increase the cross-sectional hardness variation.

In Example 1, the following process conditions could be used: Mold Temperature 168 C. and Cure Time 370 seconds. In alternate processes, the Mold Temperature could be 165 C. and the Cure Time 420 seconds.

In Example 2, the following process conditions could be used: Mold Temperature 172° C. and Cure Time 420 seconds. In alternate processes, the Mold Temperature could be 175° C. and the Cure Time 380 seconds. These conditions may vary due to the part profile. Even further variations could be observed by changing the mold temperature and cure times more drastically.

In addition to the above, the present invention provides the following advantages over the prior art: The present invention provides an orthotic giving support to the different arches under the foot. The present invention provides a replaceable orthotic, which slips in the top of the shoe and comes in direct contact with the foot or sock. The present invention can provide an orthotic which could incorporate a cushioning device and orthotic as one piece which slips in the top of the shoe. Typically 10 to 25 mm of cushion in the heel and 5 to 15 mm cushion in the forefoot can be used. The present invention provides an orthotic that is softer in thick cross-sections when compared to the thin cross-sections. The present invention provides an orthotic has a softening point of around 93 degrees Centigrade which can put in the oven for 4 minutes at this temperature, taken out of the oven, quickly placed in the shoe, and then stood on with the users foot for 1 minute to partially conform the orthotic or orthotic/midsole to the foot.

After this process is complete, it is typical to see more changes in the heel area when compared to the metatarsal area, or lateral or medial arch areas. The present invention provides a process that allows control of the hardness variation of thin cross-sections when compared to thick cross-sections of the resulting orthotic. The present invention provides an orthotic that can be custom made at different overall stiffness for sports that require different degrees of orthotic support. The present invention provides a process that allows for easily and accurately reproducible orthotics that are reproducible to the following degree (for a men's size 9 orthotic): +/−0.5 gram; +/−0.75 mm overall length;

+/−0.5 mm in the widest section; and +/−0.3 mm at the thickest section.

The present invention also provides a method allowing the production of orthotics with reproducible hardness variation to +/−5% of the hardness scale being used when cross-sectional pieces are compared. The present invention provides a process that can utilize all machines, which inject a compounded, foamable, cross-linkable, material into or onto a hot mold, which is hotter than the cross-linking and foaming temperature of the compound but the actual injection temperature of the compound is below the cross-linking and blowing temperature. The present invention provides a process that allows for injection of the compound into hot molds that are open or closed at the time of injection. The present invention provides a method of fabricating orthotics using a variety of polymers including polyolefins such as polyethylene and polypropylene; ethylene copolymers and terpolymers like ethylene vinyl acetate copolymer, ethylene methylaciylic copolymer, thermoplastic and thermosetting elastomers; polyesters such as polyethylene terephthylate and polybutylene terephthylate; as well as blends and alloys of two or more of such polymers. Polyolefins, ethylene vinyl acetate copolymers and thermosetting elastomers have been found to be particularly suitable for the instant process. The present invention provides a process that can produce orthotics as low as 0. 10 gm/cc overall density and as high as 0.90 gm/cc with typical overall part densities in the range of 0.15 gm/cc to 0.85 gm/cc.

I claim:

1. . A process for fabricating an orthotic insole of unitary construction having a hardness that is inversely proportional to the thickness thereof and adapted for use in footwear, the process comprising:
Mixing a cross-linking agent, a foaming or blowing agent to obtain a foamable cross-linkable compound;
Mixing the compound at temperatures below those that would activate the cross-linking or blowing agents;
Injecting the compound into or onto a hot mold;
Controlling the amount of the compound injected;
Controlling the speed at which the compound is injected;
Controlling the temperature at which the compound is injected;
Controlling the pressure at which the compound is injected;
Heating the compound in the mold to above the decomposition temperature of the blowing agent and above the activation temperature of the cross-linking agent;
Holding the mold closed until the cross-linking and foaming reactions take place;
Controlling the mold temperature;
Controlling the amount of time the compound is within the mold, and;
Opening the mold to allow the finished part to explode or quickly expand out of the mold.

2. A process of claim 1 wherein the cross-linking agent is at least one selected from the group consisting of peroxide, sulfur and sulfur donor cross-linking agents.

3. A process of claim 2 wherein the peroxide is at least one selected from the group consisting of Dibenzoyl peroxide, t-butyl peroxybenzoate, 1,1-Di-(t-butylperoxy)-3,3,5-trimethylcyclohexane, Dicumyl peroxide, Di-(2-t-butylperoxyisopropyl)benzene, t-butylcumylperoxide, Di-t-butylperoxide.

4. A process of claim 1 wherein the foaming agent is at least one selected from the group consisting of azobisisobutyronitrile, azodicarbonamid, p-toluene sulfonylhydrazide, 4,4′-oxybis (benzenesulfonyhydrazide), N,N-dinitrosopentamethylenetetramine and sodium bicarbonate, modified azodicarbonamids, hydrazides, and 5-phenyltetrazole.

5. A process of claim 1 wherein the foaming agent, blowing agent and cross-linking agent are mixed at a temperature of about from 90 to 140 degrees centigrade.

6. A process of claim 5 wherein the agents are mixed at a temperature of about from 95 to 120 degrees centigrade.

7. A process of claim I wherein the compound is injected into a mold having a temperature of about from 145 to 200 degrees centigrade.

8. A process of claim 7 wherein the mold has a temperature of about from 165 to 175 degrees centigrade.

9. A process of claim 1 wherein the compound is heated to a temperature of about from 145 to 200 degrees centigrade while in the mold.

10. A process of claim 9 wherein the compound is heated to a temperature of about from 165 to 175 degrees centigrade.

11. A process of claim 1 wherein the compound is injected into the mold at a pressure of about from 50 to 20,000 psi.

12. A process of claim 11 wherein the compound is injected into the mold at a pressure of about from 500 to 5000 psi.

13. A process of claim 1 wherein the compound is held within the mold for about from 180 to 600 seconds.

14. A process of claim 13 wherein the compound is held within the mold for about from 270 to 420 seconds.

15. A process of claim 1 wherein the molds are closed during injection and a vacuum assists the flow of compound into the mold through at least one vacuum port formed in an end of the mold.

16. A process of claim 1 wherein the molds are open and the compound drools onto a hot mold using a movable injector, and wherein the mold is closed after it has been filled.

17. A shoe comprising a sole and an upper connected to the sole, wherein the shoe is adapted for insertion of an orthotic according to the present invention, and wherein the shoe further comprises an orthotic made according to the process of claim 1.

18. A shoe of claim 17 wherein the orthotic further comprises a midsole and wherein the midsole and orthotic are one unitary piece.

19. An orthotic of unitary construction made according to the process of claim 1.

20. An orthotic of claim 19 wherein the hardness of the orthotic is inversely proportionate to the thickness of the orthotic.

21. An orthotic of unitary construction consisting essentially of closed cell molded foam and having a hardness that is inversely proportionate to its thickness.

22. An orthotic of claim 21 adapted to be reheated in a conventional oven for a period of time and removed, wherein the reheated orthotic is applied with pressure to an individuals foot to substantially conform to the contours of the foot.

23. An orthotic of claim 22 having metatarsal support of at least 1 mm and retaining the metatarsal support after being reheated.

24. An orthotic made according to the process of claim 1 where the orthotic further comprises a midsole.

25. An orthotic of claim 21 having a hardness of about from 25 Asker C to 90 Shore A.

26. An orthotic of claim 25 having a hardness of about from 45 Asker C to 70 Shore A.

27. An orthotic of claim 21 further comprising a cushioning layer or midsole.
